(19) United States
(12) Patent Application Publication
(10) Pub. No.: US 2020/0123276 A1
(43) Pub. Date: Apr. 23, 2020

(54) CROSS-LINKED HA BEADS AND PROCESS FOR MAKING SAME

(71) Applicant: Prollenium Medical Technologies, Inc., Aurora (CA)

(72) Inventor: Hitoshi Masui, Aurora (CA)

(21) Appl. No.: 16/661,683

(22) Filed: Oct. 23, 2019

Related U.S. Application Data
(60) Provisional application No. 62/749,400, filed on Oct. 23, 2018.

(51) Int. Cl. C08B 37/08; A61K 8/73; B01F 3/08; B01F 3/20; A61Q 19/08

(52) U.S. Cl. CPC C08B 37/0072; A61K 8/735; B01F 3/0865; A61K 2800/91; B01F 3/2014; A61Q 19/08; B01F 2003/0884; B01F 3/0803

(57) ABSTRACT

This disclosure provides a hyaluronic acid (HA) bead, a dermal filler having hyaluronic acid (HA) and a process for preparing the HA beads. The process includes combining an HA compound with sodium hydroxide, forming an HA solution, and injecting the HA solution into an oil solution, and forming an emulsion with HA beads. A cross-linking reagent is added and the mixture is stirred for 24 hours in room temperature, thereby forming cross-linked HA beads. The HA beads are cross-linked and adapted to dissolve in vivo upon contact with hyaluronidase.

### CROSS-REFERENCE TO RELATED APPLICATION

This application claims the benefit of U.S. Provisional Patent Application Serial No. 62/749,400 filed Oct. 23, 2018.

### BACKGROUND OF THE INVENTION

### Field of the Invention

The invention relates to the field of dermal fillers. More particularly, the invention relates to cross-linked hyaluronic acid beads for use in dermal fillers and a process for preparing such hyaluronic acid beads.

### 2. Prior Art.

Hyaluronic acid (HA) is a linear macromolecular polysaccharide consisting of alternately bonded β-D-N-acetylglucosamine and β-D-glucuronic acid. It is well known to inject hyaluronic acid under the dermis for cosmetic reasons. Such dermal fillers can be used to offset the effects of aging on the skin, by smoothing soft tissue defects like nasolabial folds and marionette lines as well as more substantial augmentation such as smoothing hollow cheeks resulting from lipoatrophy, or enhancing the fullness of lips.

Chemical cross-linking of HA is often necessary in order to increase the durability of the implant, since unmodified HA generally has a half-life in the dermis of only a day or two. HA with high levels of cross-linking tend to elicit a foreign body reaction, which stimulates the deposition of collagen around the foreign particle. Collagen deposition is desired if a long-lasting filler effect is sought. However, too much foreign body reaction causes inflammation and other related problems. Dermal fillers completely composed of HA with high levels of cross-linking tend to cause such problems.

Further, if an HA-based dermal filler is ever accidentally injected into a blood vessel, the blood vessel may be occluded by the dermal filler. If such an occlusion cannot be promptly removed, necrosis may result.

### SUMMARY OF THE INVENTION

Forming one aspect of the invention is a process for preparing hyaluronic acid (HA) beads, the method comprising:
combining an HA compound with sodium hydroxide, forming an HA solution; and
injecting the HA solution into an oil solution, forming an emulsion with HA beads.

In some embodiments, the process further comprises
adding a cross-linking reagent to the HA beads, forming a bead mixture; and
stirring the bead mixture for 24 hours in room temperature, thereby forming cross-linked HA beads.

Alternatively, in other embodiments, the process further comprises
adding a cross-linking reagent to the HA solution prior to the injecting; and
stirring the emulsion for 24 hours in room temperature after the injecting, thereby forming cross-linked HA beads.

Forming another aspect of the invention is a hyaluronic acid (HA) bead for use in a dermal filler, the bead being cross-linked and adapted to dissolve in vivo upon contact with hyaluronidase.

Forming another aspect of the invention is a dermal filler comprising:
hyaluronic acid with a predetermined level of cross-linking; and
hyaluronic acid beads suspended in the hyaluronic acid, the hyaluronic acid beads having a cross-linking level higher than that of the hyaluronic acid, the hyaluronic acid beads cross-linked and adapted to dissolve in vivo upon contact with hyaluronidase.

### BRIEF DESCRIPTION OF THE DRAWINGS

FIG. 1 is a collection of micrographs of HA beads according to an embodiment of the present invention degrading over time.

FIG. 2 is a flow chart illustrating a process according to an embodiment of the present invention; and

FIG. 3 is a flow chart illustrating a process according to another embodiment of the present invention.

### DETAILED DESCRIPTION OF THE EXEMPLARY EMBODIMENT

An example embodiment of an improved dermal filler, of hyaluronic acid (HA) beads for use in the dermal filler, and of processes 200, 300 for forming the HA beads will be discussed. The HA beads and dermal filler will first be described, then processes 200, 300.

### HA Beads

The hyaluronic acid beads are globules of hyaluronic acid that are cross-linked and will naturally degrade in about 9 months when injected into a patient. The HA beads are typically 80-150 microns in diameter. According to a first embodiment, the HA beads are generally spherical and translucent. According to a second embodiment, the HA beads are perfectly spherical and transparent.

The HA beads are also adapted to dissolve in vivo in 20 minutes upon contact with hyaluronidase.

A microscope enzyme degradation study was performed on the cross-linked HA beads of the present invention to demonstrate this characteristic.

In the study, 10 microliters of an HA bead suspension in phosphate buffered saline (PBS) was placed on a dimpled glass microscope slide and 100 microliters of 165 units/mL of hyaluronidase, which is also suspended in PBS, was added and mixed in with a stainless steel wire.

More PBS was added to fill the dimple in the slide and a cover slip was carefully placed over the sample to avoid the entrapment of bubbles. Mineral oil was deposited around the edges of the cover slip to seal and prevent evaporation.

The slide was placed on a temperature controlled microscope stage set to 37 Celsius and digital micrographs were acquired periodically. The magnification of the microscope was set to 25 times (the beads are 80-150 microns in diameter).

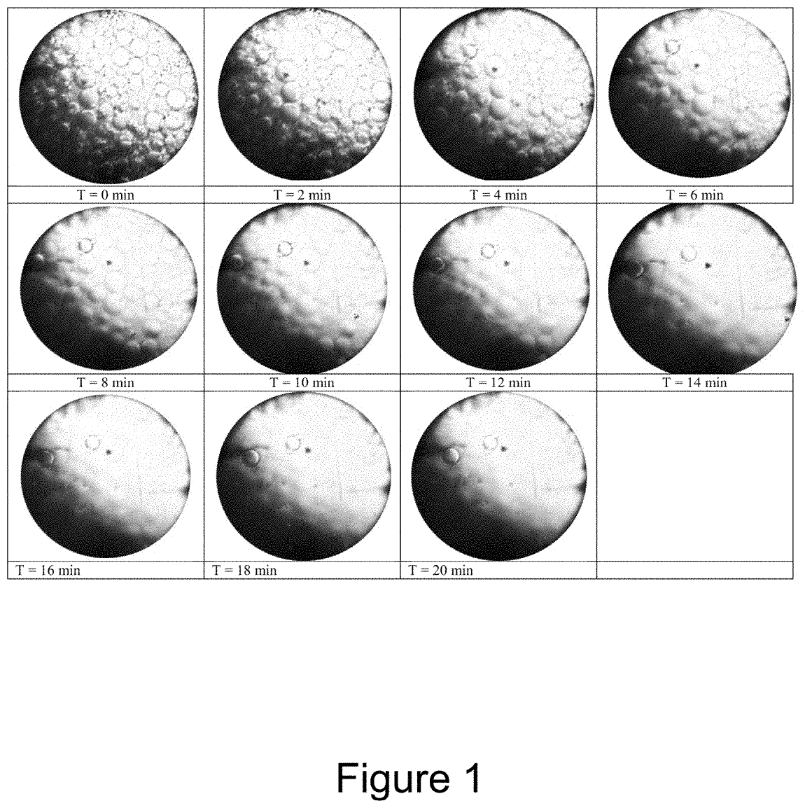

FIG. 1 is a collection of micrographs taken every 2 minutes, showing the degradation of the HA beads after 20 minutes. As can be seen at T =20 min, the HA beads have predominantly degraded and may no longer occlude the blood vessel.

Whereas a specific embodiments of the HA bead are herein shown and described, variations are possible. In some examples, the HA beads have a diameter of 1 to 1000 microns.

### Dermal Filler

The dermal filler of the present invention comprises hyaluronic acid with a predetermined level of cross-linking. The improvement of the dermal filler comprises the HA beads described above suspended in hyaluronic acid. The predetermined level of cross-linking of the hyaluronic acid is lower than the level of cross-linking of the HA beads suspended therein.

The dermal filler is made by dissolving HA in a sodium hydroxide solution. BDDE is then added and mixed into the solution. The resulting mixture is warmed and further mixed for a period of time. It is then cooled and neutralized with acid. The resulting gel mixture is diluted as appropriate and then milled into small particles. The gel particles are washed by dialysis. The HA beads, described above, and produced separately, are blended in under low shear. Whereas a specific embodiment of the dermal filler are described, variations are possible.

In some examples, rather than suspending the HA beads have a cross-linking level that is higher than the predetermined level of cross-linking of the hyaluronic acid, the HA beads would have a cross-linking level that is lower than that of the hyaluronic acid.

In some examples, rather than suspending the HA beads in hyaluronic acid, the dermal filler may be composed entirely of the cross-linked HA beads described above.

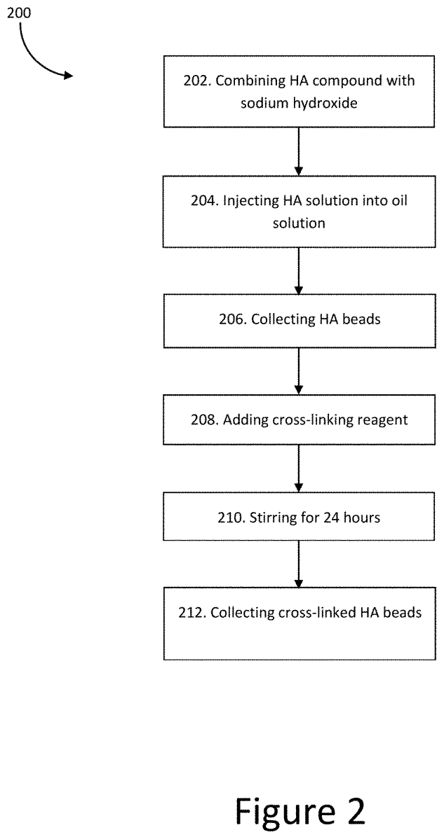

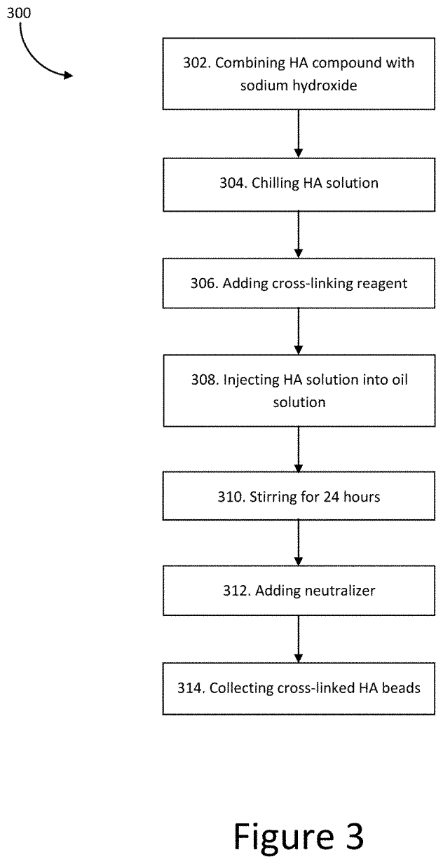

Referring to FIGS. 2 and 3, process 200 tends to form the first embodiment of the HA beads and process 300 tends to form the second embodiment of the HA beads.

### Process 200

FIG. 2 is a chart illustrating process 200 for preparing cross-linked HA beads. At 202, sodium hyaluronate (NaHA) is combined with 1% sodium hydroxide, to form an HA solution with a concentration of 80 mg/mL of NaHA.

At 204, one milliliter of the HA solution is injected into 5 mL of a stirred oil solution using a 22 gauge syringe needle at a rate of 1 mL/min. The oil solution comprises an emulsifier, 2% Tween-80, in isobutanol and is stirred at 1500 rotations per minute with an overhead stirrer having an anchor shaped paddle. The stirring is maintained for 1 minute after the HA solution is injected into the oil solution. The separation of the HA solution and oil solution forms an emulsion with HA beads.

While the emulsion is stirred at 204, the emulsion is drained into 50 mL of another stirred solution containing 2% Tween-80 in dry ethanol, the dry ethanol being stirred at 1100 rotations per minute with a magnetic stirrer. This ethanol mixture is then stirred for at least another five minutes.

At 206, the resulting HA beads are collected by vacuum filtration through a sintered glass and fritted funnel. The HA beads are then washed multiple times with ethanol, washed once with hexanes, and then dried.

At 208, the HA beads are cross-linked. The isolated HA beads are suspended in dry ethanol, and a stoichiometric amount of potassium hydroxide is added along with a cross-linking reagent forming a bead mixture. In this case, 1,4-butanediol diglycidyl ether (BDDE) is added in a BDDE:HA mass ratio of 1:5.

At 210, the bead mixture is stirred for 24 hours at room temperature, thereby cross-linking the HA beads.

The cross-linked HA beads are collected at 212 as before, by vacuum filtration through a sintered glass and fritted funnel. The cross-linked beads are further washed multiple times with ethanol, washed once with hexanes, and then dried.

Whereas a specific embodiment of process 200 is described, variations are possible.

In some examples, at 204, rather than draining the emulsion into 50 mL of a dry ethanol solution, the emulsion may be drained into a solution containing a different alcohol, such as methanol, propanol, or isopropanol.

Further, in other examples, at 206 and/or 212, the HA beads and/or cross-linked HA beads may be washed with a different alcohol, such as methanol, propanol, or isopropanol, and then washed once with a volatile lipophilic solvent other than hexane.

### Process 300

FIG. 3 is a chart illustrating process 300 for preparing cross-linked HA beads. At 302, 300 mg of hyaluronate acid is combined or dissolved in 3.00 mL of 1% sodium hydroxide, forming an HA solution.

At 304, the HA solution is chilled in an ice bath.

While chilling the HA solution, 300 μL of a cross-linking reagent, BDDE in this case, is mixed well into the HA solution at 306.

At 308, the resulting HA solution with BDDE is then injected into 15.0 mL of a stirred oil solution using a 16-gauge needle at a rate of 1 mL/min. The oil solution comprises an emulsifier, 5% SPAN-80, in heavy mineral oil and is stirred at 800 rotations per minute. The separation of the HA solution and oil solution forms an emulsion with HA beads.

At 310, the emulsion is stirred for 24 hours at room temperature, thereby cross-linking the HA beads.

After the stirring step, at 312, 3.00 mL of a neutralizer is added to the emulsion. The neutralizer has a composition as set out below:

| Cmpd | g or mL per L | g or mL |
|---|---|---|
| Water | 920 | 230 |
| HCl | 41.67 | 10.4167 |
| Na2HPO4 | 7.61 | 1.9018 |
| KCl | 1.06 | 0.2641 |
| NaCl | 27.65 | 6.9125 |
| KH2PO4 | 1.27 | 0.3170 |

The cross-linking reaction is performed at very high pH. After cross-linking, the neutralizer lowers the pH to physiological levels and helps to stop any further reaction with unreacted cross-linking material.

The mixture is further stirred for another 30 minutes and then 5 mL of hexanes is added in preparation for the collecting and washing steps.

The cross-linked HA beads are collected at 314, by vacuum filtration using a coarse sintered glass Buchner funnel. The cross-linked HA beads are further rinsed multiple times with hexanes and air dried to remove most of the hexanes without drying out the beads.

The cross-linked HA beads are then washed once more with phosphate-buffered saline (PBS) and then dried.

Whereas a specific embodiment of process 300 is described, variations are possible.

In some examples, at 314, the cross-linked HA beads may be washed with a volatile lipophilic solvent other than hexane.

In some examples, at 312, a neutralizer having a different composition may be used.

### Advantages

An advantage of the present invention is that the HA beads can have an independent level of cross-linking relative to their surrounding substance. As such, when suspended in hyaluronic acid, the HA beads can have a cross-linking level different from that of the hyaluronic acid. As noted in the background, hyaluronic acid with high levels of cross-linking tend to elicit a foreign body reaction, which stimulates the deposition of collagen around the foreign particle. However, too much foreign body reaction causes inflammation and other related problems.

By suspending cross-linked HA beads with a high level of cross-linking in an HA dermal filler with low levels of cross-linking, for example, it will be possible to finely “tune” the dermal filler for a desired amount of foreign body reaction by tweaking the ratio of HA beads to hyaluronic acid in the dermal filler.

Further, having a dispersion of highly cross-linked HA beads will allow the individual beads to be individually encapsulated with collagen rather than having a bolus of highly cross-linked gel being encapsulated. In such cases, collagen would tend to form around the periphery of the bolus.

An advantage of the dermal filler that is entirely comprised of HA beads is that it helps to illicit less foreign body response. It was found that the solid, smooth, spherical particles of the described HA beads illicit less foreign body response than particles having sharp edges. The current milling process, which produces the gel particles for the base hyaluronic acid, creates gel particles with sharp edges.

Another advantage of the present invention is that the HA beads can be degraded in vivo within 20 minutes upon contact with a hyaluronidase enzyme.

The presently claimed cross-linked HA beads will naturally degrade in about 9 months when injected into a patient. However, if the HA beads are accidentally injected into a blood vessel, occlusion may result. The HA beads and the dermal filler in the present invention, can be promptly dissolved by injecting a hyaluronidase enzyme solution, which is available by prescription, into the patient.

Whereas a specific embodiments of the HA bead, dermal filler and processes of making same are herein shown and described, variations are possible.

Accordingly, the invention should be understood to be limited only by the accompanying claims, purposively construed.

What is claimed is:

1. A process for preparing hyaluronic acid (HA) beads, the process comprising:
combining an HA compound with sodium hydroxide, forming an HA solution; and
injecting the HA solution into an oil solution, forming an emulsion with HA beads.

2. The process of claim 1, further comprising:
adding a cross-linking reagent to the HA beads, forming a bead mixture; and
stirring the bead mixture for 24 hours in room temperature, thereby forming cross-linked HA beads.

3. The process of claim 2, wherein the HA solution is injected into the oil solution using a syringe needle.

4. The process of claim 3, wherein the oil solution is stirred as the HA solution is injected.

5. The process of claim 4, wherein the oil solution comprises 2% Tween-80 in isobutanol.

6. The process of claim 5, further comprises removing and cleaning the HA beads from the emulsion prior to the adding of the cross-linking reagent.

7. The process of claim 6, wherein the cross-linking reagent is added to the HA beads in a reagent:HA mass ratio of 1:5.

8. The process of claim 1, further comprising:
adding a cross-linking reagent to the HA solution prior to the injecting step; and
stirring the emulsion for 24 hours in room temperature after the injecting step, thereby forming cross-linked HA beads.

9. The process of claim 8, further comprising chilling the HA solution in an ice bath after the dissolving step.

10. The process of claim 9, wherein the HA solution is injected into the oil solution using a syringe needle.

11. The process of claim 10, wherein the oil solution is stirred as the HA solution is injected therein.

12. The process of claim 11, wherein the oil solution comprises 5% SPAN-80 in mineral oil.

13. The process of claim 12, further comprising adding a neutralizer to the emulsion after the stirring step.

14. A hyaluronic acid (HA) bead for use in a dermal filler, the bead being cross-linked and adapted to dissolve in vivo upon contact with hyaluronidase.

15. The HA bead of claim 14 adapted to dissolve in vivo in 20 minutes upon contact with the hyaluronidase.

16. The HA bead of claim 15 having a generally spherical shape.

17. The HA bead of claim 16 having a diameter of 1 to 1000 microns.

18. A dermal filler comprising:
hyaluronic acid with a predetermined level of cross-linking; and
hyaluronic acid beads suspended in the hyaluronic acid, the hyaluronic acid beads having a cross-linking level higher than that of the hyaluronic acid, the hyaluronic acid beads cross-linked and adapted to dissolve in vivo upon contact with hyaluronidase.

19. The dermal filler of claim 18, wherein the hyaluronic acid beads are cross-linked and adapted to dissolve in vivo in 20 minutes upon contact with the hyaluronidase.

20. The dermal filler of claim 19, wherein the hyaluronic acid beads have a diameter of 1 to 1000 microns.
